(19) United States
(12) Patent Application Publication
(10) Pub. No.: US 2025/0327031 A1
(43) Pub. Date: Oct. 23, 2025

(54) MEDIA, KITS AND METHODS FOR DIFFERENTIATING TENOCYTES OR CHONDROCYTES

(71) Applicant: STEMCELL TECHNOLOGIES CANADA INC., Vancouver (CA)

(72) Inventors: Arthur SAMPAIO, Vancouver (CA); Alessandro DEI, Vancouver (CA)

(21) Appl. No.: 18/873,367

(22) PCT Filed: Jun. 29, 2023
(86) PCT No.: PCT/CA2023/050895

§ 371 Date: Dec. 10, 2024

Related U.S. Application Data
(60) Provisional application No. 63/357,104, filed on Jun. 30, 2022.

(51) Int. Cl. C12N 5/077

(52) U.S. Cl. CPC C12N 5/066; C12N 2501/15; C12N 2501/19; C12N 2506/02; C12N 2506/03; C12N 2506/45

(57) ABSTRACT

Disclosed herein are media, kits, and methods for differentiating mammalian progenitor cells to a downstream lineage of cells. Embodiments of disclosed media, methods and kits may be used to differentiate mammalian progenitor cells to tenocytes or tenocyte-like cells, or chondrocytes or chondrocyte-like cells.

### CROSS REFERENCE TO RELATED APPLICATION

This application claims the benefit of U.S. Provisional Patent Application No. 63/357,104, filed Jun. 30, 2022, the entire content of which is hereby incorporated by reference in its entirety.

### TECHNICAL FIELD

This disclosure relates to cell culture applications, and more specifically to cell culture applications using mammalian progenitor cells, and still more specifically to cell culture applications differentiating mammalian progenitor cells to downstream cell lineage(s).

### BACKGROUND

With over 30 million tendon-related surgical procedures taking place annually, tendon injuries are one of the most frequent human orthopaedic ailments.

Due to the scarce cellularity and vascularization of tendon tissue, the physiological tendon healing process is slow and imperfect. Moreover, current strategies to treat tendon injuries involve lengthy rehabilitation periods and often result in deposition of fibrotic scar tissue and ectopic ossification. Thus, alternative solutions to promote tendon healing and regeneration are important and, recently, cell-based therapies have emerged as a promising strategy.

Multipotent mesenchymal stromal cells (MSCs) and pluripotent stem cells (PSCs) are attractive candidates for cell-based therapies because of their self-renewal and differentiation capacity, immunomodulatory properties, and the ease of isolation and manipulation. However, the translation of cell-based therapies from bench to bedside are hindered by the use of animal-derived supplements during in vitro culture, and by the lack of standardized protocols for in vitro differentiation of stem cells toward the tenogenic and other lineage(s).

Therefore, there is a need for robust and efficient media, kits, supplements, and methods to differentiate mammalian MSCs and PSC-derived mesenchymal progenitor cells to downstream cell lineages, such as tenocytes or tenocyte-like cells (TLCs).

### SUMMARY

The present disclosure relates to methods, culture media, supplements, and kits for differentiating mammalian progenitor cells to the tenogenic lineage. In one embodiment, this disclosure relates to in vitro differentiation methods, and cell culture media, supplements, and kits for in vitro applications.

In one aspect of this disclosure are provided methods for differentiating mammalian progenitor cells to the tenogenic lineage. The methods may comprising culturing the mammalian progenitor cells in a first medium comprising a basal medium and at least one first growth factor. The methods may further comprise culturing the arising cells in a second medium comprising a basal medium and at least one second growth factor.

In one embodiment, the at least one first growth factor and the at least one second growth factor are transforming growth factor (TGF) superfamily members. In one embodiment, the at least one first growth factor is different from the at least one second growth factor.

In one embodiment, the at least one first growth factor is an agonist of TGF-β signaling. In one embodiment, the agonist of TGF-β signaling is one or more of TGF-β1, TGF-β2, or TGF-β3. In one embodiment, the agonist of TGF-β signaling includes at least TGF-β3.

In one embodiment, the at least one second growth factor is an agonist of BMP signaling. In one embodiment, the agonist of BMP signaling is GDF-5, GDF-6, GDF-7, or GDF-8. In one embodiment, the agonist of BMP signaling is GDF-5.

In another aspect of this disclosure are provided methods for differentiating mammalian progenitor cells to a downstream lineage of cells. The methods may comprise culturing the mammalian progenitor cells in a basal medium comprising at least one first growth factor, and optionally at least one second growth factor.

In one embodiment, the downstream lineage of cells are chondrocytes or chondrocyte-like cells. In one embodiment, the methods may further comprise deriving the chondrocytes or chondrocyte-like cells when the basal medium comprises the at least one first growth factor and the at least one second growth factor.

In one embodiment, the downstream lineage of cells are tenocytes or tenocyte-like cells. In one embodiment, the methods may further comprise deriving the tenocytes or tenocyte-like cells in the presence of at least one first growth factor. In one embodiment, the methods may further comprise deriving the tenocytes or tenocyte-like cells in the presence of at least one second growth factor sequentially after exposure to the at least one first growth factor.

In another aspect of this disclosure are provided methods for differentiating mammalian progenitor cells to a downstream lineage of cells. The methods may comprise generating a fated cell population by contacting the mammalian progenitor cells with a first medium. In one embodiment, the first medium comprises a basal medium and at least one first growth factor (e.g TGF superfamily agonist). In one embodiment, the fated cell population may be matured into the downstream lineage of cells by continued culture in the first medium.

In one embodiment, the methods may further comprise biasing the fated cell population to the downstream lineage of cells by contacting the fated cell population with a second medium. In one embodiment, the second medium comprises a basal medium and at least one second growth factor (e.g TGF superfamily agonist).

In one embodiment, the TGF superfamily agonist is the same in the first and second media. In one embodiment, the TGF superfamily agonist is different in the first and second media.

In one embodiment, the TGF superfamily agonist is an agonist of TGF-β signaling. In one embodiment, an agonist of TGF-β signaling is one or more of TGF-β1, TGF-β2, or TGF-β3. In one embodiment, an agonist of TGF-βsignaling is at least TGF-β3. In one embodiment, an agonist(s) of TGF-βsignaling is comprised in the first medium.

In one embodiment, the TGF superfamily agonist is an agonist of BMP signaling. In one embodiment, an agonist of BMP signaling is a GDF, such as GDF-5. In one embodiment, an agonist of BMP signaling is GDF-5, GDF-6, GDF-7, or GDF-8. In one embodiment, an agonist of BMP signaling is GDF-5. In one embodiment, an agonist of BMP signaling is comprised in a first and/or second medium.

In one embodiment, a TGF superfamily agonist in a first medium is one or more of TGFβ1, TGF-β2, or TGF-β3, preferably comprising TGF-β3, and a TGF superfamily agonist in a second medium is an agonist of BMP signaling (e.g. a GDF, such as GDF-5).

In one embodiment, a first medium does not contain an agonist of BMP signaling and a second medium does not contain an agonist of TGF signaling. In one embodiment, a first medium and a second medium contains an agonist of BMP signaling (e.g. a GDF, such as GDF-5), and only the first or neither the first and second media contain an agonist of TGF-β signaling. In one embodiment, only a single medium is used, comprising a basal medium and one or both of an agonist of BMP signaling (e.g. a GDF, such as GDF-5) and an agonist of TGF-β signaling.

In one embodiment, the downstream lineage of cells are tenocytes or tenocyte-like cells. In one embodiment, the downstream lineage of cells are chondrocytes or chondrocyte-like cells.

In one embodiment, the methods may further comprise withdrawing the fated population of cells from contact with a first medium prior to the biasing step.

In the methods disclosed herein, the mammalian progenitor cells may be MSC, BM-MSCs, PSC, mesenchymal progenitor cells, MPC, ADSC, or tendon-derived cells (TDC). In one embodiment, the PSC are iPSCs or ESCs.

In one embodiment, the mammalian progenitor cells are PSC-derived. In one embodiment, the PSC-derived mammalian progenitor cells are PSC-derived MSC, or are PSC-derived MPCs or PSC-derived mesenchymal progenitor cells.

In one embodiment, the mammalian progenitor cells are of human origin, equine origin, rodent origin, porcine origin, bovine origin or canine origin.

In the methods disclosed herein, differentiation may be under serum-free conditions. In the methods disclosed herein, differentiation may be under ACF conditions.

In another aspect of this disclosure are provided media and/or kits for differentiating mammalian progenitor cells to a downstream lineage of cells. Media and or kits may comprise a basal medium and a first supplement to be added thereto.

In one embodiment, media and kits may further comprise a second supplement to be added to a basal medium. First and/or second supplements may comprise one or more TGF superfamily members. Where media and kits comprise first and second supplements the one or more TGF superfamily members may be the same or different. In one embodiment, the TGF superfamily member(s) may be selected from a TGF signaling agonist (e.g. an agonist of TGF-β signaling, such as TGF-β1, TGF-β2, or TGF-β3) and a BMP signaling agonist (e.g. a GDF, such as GDF-5, GDF-6, GDF-7, or GDF-8).

In another aspect of this disclosure, are provided media and methods for expanding primary mammalian (e.g. human) tenocytes under serum-free and/or ACF conditions. In one embodiment, such media and methods sustain increased population doublings and/or prolonged culture duration compared to conventional, undefined (bovine) serum-containing conditions.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 shows the effects of temporal growth factor addition on the differentiation of MSCs. TGF-β3 (TM-1) and GDF-5 (TM-2) were added to culture media either individually, simultaneously, or sequentially, and the resulting cultures were analyzed by time-course qRT-PCR for tenogenic marker expression. The control (“CTRL”) relates to maintenance/expansion culture conditions of MSCs in a basal medium of this disclosure: MesenCult ACF+, “MACF+” (A). Representative immunofluorescence images at 40× magnification of cultures stained for TNMD (B), and the percentage of elongated spindle-shaped cells was quantified and plotted (C). Representative immunofluorescence images at 10× magnification of cultures stained for collagen type I, type III, type II, and aggrecan as denoted by arrowheads (D), and plots showing the relative quantification of immunofluorescence staining of collagen type II and aggrecan proteins (E). Gene expression levels of chondrogenic markers among MSCs cultured simultaneously in TM-1+TM-2 (F). Bars indicate the mean of replicates, and asterisks indicate statistically significant differences at * p<0.05, ** p<0.01, *** p<0.001. Scale bars: 100 μm and 50 μm in 10× and 40× immunofluorescence images, respectively.

FIG. 2 shows differentiation efficiency of human bone marrow-derived MSCs (hBM-MSCs), human adipose-derived stem cells (hADSCs) and human tendon-derived cells (hTDCs) towards tenogenic lineage. Time-course qRT-PCR analysis of tenogenic marker expression on days 0, 3, 8 and 21 of differentiation in TM-1>TM-2 (A). Representative immunofluorescence images at 10× magnification of collagen type I, type II, type III, and aggrecan expression, as denoted by arrowheads (B), and at 40× magnification of SCX and TNMD expression at day 3 and day 21 (C) for cells in control (hBM-MSCs and hADSCs in MACF+; hTDCs in DMEM+10% FBS) or TM-1>TM-2 conditions. Scale bars: 100 μm and 50 μm in 10× and 40× immunofluorescence images, respectively. Histograms (D) and dot plot (E) summarize the flow cytometry analysis of TNMD expression in day 21 cultures. Each point on the dot plot represents a biological replicate (n=4 for hBM-MSCs, n=4 for hADSCs, n=3 for hTDCs) +/−SD.

FIG. 3 shows in-vitro expansion of primary human tenocytes for up to 10 passages in FBS-based standard culture conditions and in a serum-free and ACF basal medium (MACF+). Bar graph showing population doublings at each passage (A), and a growth curve showing population doublings over >50 days in culture (B). Bars indicate mean of replicates +/−SD, and asterisks indicate statistically significant differences at * p<0.05, ** p<0.01, *** p<0.001.

FIG. 4 summarizes the expression of various markers during the derivation of PSC-derived mesenchymal progenitor cells. Heat maps reflect expression levels of mesoderm (TBXT, NCAM, and MIXL1) and pluripotency (OCT4, SOX2, NANOG, and EPCAM1) markers during derivation of early mesoderm progenitors from hiPSCs (A) and hESCs (B) after a 21-day differentiation protocol. Bar graphs summarize flow cytometry analyses evaluating the purity of MPCs obtained from hiPSCs (C) and hESCs (D) after a 21-day differentiation protocol.

FIG. 5 summarizes marker expression during differentiation of PSC-derived MPCs into cells with tenocyte-like properties. Heat maps reflect qRT-PCR analysis of tenogenic markers in hiPSCs (A) and hESCs (B) during differentiation as described herein. Representative 2× BF images of Sirius Red staining at the start (day 21) and end (day 42) of MPC differentiation derived from different PSC (1C, FO16, and FO22) and hESC (H1, HES3) lines (C). Scale bars show 1000 μm. Representative immunofluorescence images at 10× magnification after staining for collagen type I, type II, type III, and aggrecan (as denoted by arrowheads) at the end of differentiation (D). Scale bars show 100 μm. Histograms of TNMD expression levels after (day 42) differentiation of hiPSC-derived (E) and hESC-derived (F) tenocyte-like cells.

FIG. 6 summarizes RNA-seq data of the hMSC-or hPSC-derived tenocyte-like cell transcriptomes. Bar graphs show differential gene expression analysis comparing the transcriptomes of MSC-derived tenocytes (A) and PSC-derived tenocyte-like cells (B) to control transcriptomes (TDCs, MSCs, or PSCs, as shown). Bar graphs of the gene set enrichment analysis in MSC-derived (C) and PSC-derived (D) tenocytes relative to controls.

FIG. 7 summarizes RNA-seq data comparing the transcriptomes of various cell populations. Principal component analysis (PCA) statistically clustered different cell types into each quadrant of the graph (A). Bar graphs in (B) and (C) indicate the number of differentially expressed genes between cell populations differentiated from either hMSCs or hPSCs using media and methods of this disclosure. Bar graphs plot the most enriched gene set enrichment analysis hits when comparing MSC-derived tenocytes with PSC-derived tenocyte-like cells (D).

FIG. 8 shows a heat map comparing the transcriptomes of different cell types. Shown is the relative expression of a panel of 50 genes related to tendon development, tenogenic differentiation, and tendon extracellular matrix (ECM) components among the indicated cell types.

FIG. 9 shows tenogenic differentiation of equine MSCs and superficial digital flexor tendons (SDFTs). Representative 2× BF images of eMSCs and eSDFTs cultures stained with Sirius Red at the start (day 0) and end (day 21) of tenogenic differentiation (A). Scale bars show 1000 μm. Representative immunofluorescence images at 40× magnification of tenogenic cultures stained for TNMD (red) at day 21 after differentiation of eMSCs and eSDFTs (B). Histograms summarize flow cytometry data of TNMD expression in day 21 cultures (C).

### DETAILED DESCRIPTION

This disclosure relates to media compositions and/or media supplements and/or kits comprising media and supplements, and to methods for differentiating mammalian progenitor cells to a downstream lineage of cells. In one embodiment, this disclosure relates to differentiating mammalian progenitor cells to the tenogenic lineage in media or by methods of this disclosure.

Where used in this disclosure, the term “tenogenic lineage” or “tenocyte-like cells” refers to a population of cells exhibiting characteristics of tenocytes or tenocyte progenitors. Cells of the tenogenic lineage or tenocyte-like cells may be differentiated from MSCs, PSCs, or multipotent cells, or may be isolated from a subject. Characteristics of tenocytes or tenocyte progenitors may include lineage-specific gene expression, an elongated spindled morphology, and/or production of extracellular matrix comprising appropriate proteins. Examples of lineage-specific genes may include one or more of SCX, MKX, TNMD, COL1A1, and COL3A1. Tenogenic lineage cells may also express markers of the muscle lineage and/or adipocyte lineage.

Where used in this disclosure, the term “chondrogenic lineage” or “chondrocyte-like cell(s)” refers to a population of cells exhibiting characteristics of chondrocytes or chondrocyte progenitors. Cells of the chondrogenic lineage or chondrocyte-like cells may be differentiated from MSCs, PSCs, or multipotent cells. Characteristics of chondrocytes or chondrocyte progenitors may include expression of lineage-specific genes, a flattened morphology, and/or production of extracellular matrix comprising appropriate proteins. Chondrogenic differentiation of pluripotent or multipotent cells may be evaluated by the measurement of the pellet volume, cell morphology and matrix composition by hematoxylin-eosin (HE), safran and alcian blue staining.

Where used in this disclosure, the term “MSC” refers to multipotent cells capable of self-renewal and of differentiating into various cell types including osteoblasts, chondrocytes, adipocytes, tenocytes, myotubes, neural cells, and hematopoietic-supporting stroma cells. MSCs are localized in bone marrow, but may be isolated from most tissues of the body including, but not limited to, compact bone, adipose tissue, cord blood, peripheral blood, muscle, tendon, cartilage, hair, scalp tissue, fetal liver and lung. MSCs, or rather MSC-like cells, may be differentiated from PSCs, such as induced PSC (iPSCs) or embryonic stem cells (ESC), by directed differentiation or forward programming (e.g. a mesodermal precursor cell, MPC).

Where used in this disclosure, the term “mammalian progenitor cell” refers to a cell or cell type capable of differentiating to one or more lineages downstream of MSC, such as to or toward the tenogenic lineage and/or the chondrogenic lineage. In one embodiment, a mammalian progenitor cell is intermediate a pluripotent or totipotent cell and the tenogenic lineage and/or the chondrogenic lineage. In one embodiment, a mammalian progenitor cell is a PSC, such as iPSC or ESC. In one embodiment, a mammalian progenitor cell is a MSC, an ADSC, a hematopoietic stem cell, or a progenitor thereof. In one embodiment, a mammalian progenitor cell may be PSC-derived, such as a PSC-derived MSC, a MSC-like cell, or a MPC.

Where used in this disclosure, the term “animal component-free (ACF)” refers to conditions, workflows, and/or formulations that do not contain at minimum primary raw materials derived directly from animal (including human) tissue or body fluid.

### Media and Kits

In one aspect of this disclosure are provided media (and supplements to be added to media) for differentiating (a population of) PSC or mammalian progenitor cells to a downstream lineage, such as the tenogenic lineage or the chondrogenic lineage. In some embodiments, media of this disclosure are comprised in a system or a kit, comprising at least a first medium. In some embodiments, media of this disclosure are comprised in a system or a kit, comprising media for different stages of differentiation (e.g. stage-specific media).

Mammalian progenitor cells to be cultured in or contacted by media of this disclosure are not particularly limited. Thus, mammalian progenitor cells may correspond to any cell type that may be differentiated to a lineage of interest, such as the tenogenic and/or chondrogenic lineages. Differentiation efficiencies may be enhanced if the mammalian progenitor cells and the target cell of differentiation share a common germ layer origin. For example, if targeting a tenocyte (or tenocyte-like cell) or chondrocyte (or chondrocyte-like cell), then the mammalian progenitor cell may be a type of upstream mesodermal or mesenchymal stem or progenitor cell.

In certain embodiments, mammalian progenitor cells may be of human, equine, bovine, porcine, rodent, or canine origin.

By way of non-limiting example mammalian progenitor cells may be MSCs, bone marrow-derived MSCs (hBM-MSCs), mesenchymal progenitor cells, adipose tissue-derived stromal cells (ADSCs), tendon-derived cells (TDCs), or tendon progenitor stem cells (TPSCs). In certain embodiments, mammalian progenitor cells are PSC-derived, including but not limited to PSC-derived MSC, PSC-derived mesenchymal progenitor cells, or PSC-derived MPCs.

Media of this disclosure for differentiating mammalian progenitor cells may comprise a single, first medium. In one embodiment, a first medium comprises a basal medium and at least one first growth factor. A first medium may be a complete formulation or may be provided as a basal medium to be combined with one or more supplements.

Media of this disclosure for differentiating mammalian progenitor cells may comprise a first medium and a second medium for stage-specific differentiation. In one embodiment, first and second media are complete formulations. In one embodiment, first and second media are provided as a basal medium (whether a common basal medium or different basal media) to be combined with one or more supplements, such as a first supplement for differentiating mammalian progenitor cells to an intermediate stage and a second supplement for differentiating the intermediate stage cells to a cell type of interest.

A basal medium may be any medium that is capable of supporting the differentiation of mammalian progenitor cells to a target downstream lineage of cells. In one embodiment, a basal medium supports the expansion or differentiation of MSC-and/or PSC-derived tenogenic (e.g. tenocytes or tenocyte-like cells) or chondrogenic (e.g. chondrocytes or chondrocyte-like cells) cells. Basal media are known in the art, such as RPMI, DMEM, F-12, MCDB153, DMEM/F-12, Adv DMEM, Adv DMEM/F-12, STEMdiff™ branded or MesenCult™ branded media. Basal media typically include carbohydrates, amino acids, trace elements, lipids, proteins, buffers, salts, and the like. In some embodiments, basal media do not include one or more of the foregoing types of components, which may be comprised in a supplement to be added thereto.

With reference to a first medium of this disclosure, the at least one first growth factor comprised therein is not particularly limited, as long as it facilitates the differentiation or fating of a mammalian progenitor cell, whether to a cell of interest or to a cell type intermediate the mammalian progenitor cell and the cell type of interest.

In one embodiment, the at least one first growth factor is a transforming growth factor (TGF) superfamily agonist, such as one or more agonist of TGF signaling and/or an agonist of BMP signaling. In one embodiment, an agonist of TGF signaling and/or an agonist of BMP signaling is a protein or a small molecule.

In one embodiment, an agonist of TGF signaling is an agonist of TGF-β signaling. In one embodiment, an agonist of TGF-β signaling is one of or one or more of TGF-β1, TGF-β2, or TGF-β3. In one embodiment, an agonist of TGF-β signaling is TGF-β3.

In some embodiments, a concentration of a TGF superfamily agonist (e.g. TGF-β signaling agonist) is between about 0.1 ng/ml and 100 μg/ml, between about 0.5 ng/ml and 10 μg/ml, between about 1 ng/ml and 1 μg/ml, between about 2 ng/ml and 500 ng/ml, between about 3 ng/ml and 100 ng/ml, or between about 5 ng/ml and 10 ng/ml.

In some embodiments, a concentration of TGF-β3 in a first medium (or supplement) is between about 0.1 ng/ml and 100 μg/ml, between about 0.5 ng/ml and 10 μg/ml, between about 1 ng/ml and 1 μg/ml, between about 2 ng/ml and 500 ng/ml, between about 3 ng/ml and 100 ng/ml, or between about 5 ng/ml and 10 ng/ml.

In some embodiments, a concentration of an agonist of BMP signaling (e.g GDF-5) is between about 0.1 ng/ml and 100 μg/ml, between about 0.5 ng/ml and 10 μg/ml, between about 1 ng/ml and 1 μg/ml, between about 2 ng/ml and 500 ng/ml, between about 3 ng/ml and 100 ng/ml, or between about 5 ng/ml and 20 ng/ml.

With reference to a second medium of this disclosure, an at least one second growth factor comprised therein is not particularly limited, as long as it facilitates differentiating or biasing an intermediate cell population (e.g. a cell-type downstream of mammalian progenitor cells) to a cell of interest.

In one embodiment, an at least one first growth factor and an at least one second growth factor are TGF superfamily members. In one embodiment, an at least one first growth factor and an at least one second growth factor are the same TGF superfamily members. In one embodiment, an at least one first growth factor and an at least one second growth factor are different TGF superfamily members. In one embodiment, an at least one second growth factor (e.g. agonist of BMP signaling) is not comprised in a first medium. In the same or different embodiment as the foregoing, an at least one first growth factor (e.g. agonist of TGF signaling) is not comprised in a second (or any) medium of this disclosure.

In one embodiment, an at least one second TGF superfamily member is an agonist of BMP signaling. In one embodiment, an agonist of BMP signaling is a protein or a small molecule. In one embodiment, an agonist of BMP signaling is a growth differentiation factor (GDF) family member. In one embodiment, the GDF family member is GDF-5, GDF-6, GDF-7, or GDF-8. In one embodiment, the GDF family member is GDF-5.

In some embodiments, a concentration of an agonist of BMP signaling (e.g GDF-5) is between about 0.1 ng/ml and 100 μg/ml, between about 0.5 ng/ml and 10 μg/ml, between about 1 ng/ml and 1 μg/ml, between about 2 ng/ml and 500 ng/ml, between about 3 ng/ml and 100 ng/ml, or between about 5 ng/ml and 20 ng/ml.

Depending on the differentiation desired, exposure of mammalian progenitor cells (or an intermediate population downstream thereof) to media of this disclosure and growth factors contained therein may be varied. In one embodiment, mammalian progenitor cells are exposed to a first medium of this disclosure (comprising at least one first growth factor) and then the arising cells are exposed to a second medium of this disclosure (comprising at least one second growth factor). This may be the case when differentiating tenocytes, or tenocyte-like cells, from a mammalian progenitor cell, for example. In such an embodiment, a first medium comprising one or more agonist of TGF-β signaling contacts the mammalian progenitor cells, and then the arising cells are contacted with a second medium comprising an agonist of BMP signaling. Thus, in such an embodiment, the at least one first growth factor and at least one second growth factor are used sequentially. In one embodiment, tenocytes or tenocyte-like cells are differentiated from a mammalian progenitor cell in only a culture medium comprising an agonist of BMP signaling (without prior or concomitant exposure to a different agonist of TGF signaling). In embodiments differentiating chondrocytes (or chondrocyte-like cells) from a mammalian progenitor cell, a medium may comprise both at least one first growth factor and at least one second growth factor.

In one embodiment, the at least one first growth factor (e.g. agonist of TGF-β or BMP signaling) induces SCX expression, thereby generating cells fated for the tenogenic lineage. In one embodiment, the at least one second growth factor (e.g. agonist of BMP signaling) induces TNMD expression, thereby generating cells biased for the tenogenic lineage. In one embodiment, the at least one second growth factor (e.g. GDF-5) induces SCX and TNMD expression.

Media of the present disclosure may also be supplemented with other proteins, growth factors, and/or small molecules that support tenogenic or chondrogenic differentiation.

In one embodiment, media of this disclosure are for in-vitro differentiation of mammalian progenitor cells to a downstream lineage of cells. In one embodiment, media of this disclosure may be applied to a dressing to be placed on a subject in need, and thus media of this disclosure may differentiate mammalian progenitor cells to a downstream lineage of cells in vivo, such as to treat an ailment, illness, or disease in the subject, or for cosmetic or non-therapeutic purposes.

In one aspect, a cell composition produced in media of this disclosure (e.g. in vitro) may be used to treat an ailment, illness, or disease in subject in need, or for cosmetic or non-therapeutic purposes, by administering the composition at a site of interest, such as on a dressing, scaffold, substrate, and/or matrix. By way of example, the foregoing uses may relate to tendon or cartilage repair or regeneration. Such compositions, whether comprising MSCs or PSCs induced toward tenocyte or chondrocyte lineages, may take the form of a (micro) tissue or a cell suspension. In one embodiment, a composition may be used (in a mammal) to treat, repair or replace defects and/or injury to tissues, such as skin, muscle, tendon, cartilage, or ligament.

Media of this disclosure may support serum-free and/or ACF workflows.

In one embodiment, an agonist of BMP signaling is selected based on the downstream lineage of cells desired. In one embodiment, an agonist of BMP signaling may be GDF-5 (if not applied after TGF-β3), BMP-2 or BMP-4 when the downstream lineage of cells are chondrocytes or chondrocyte-like cells.

In one aspect, the present disclosure also provides kits for differentiating mammalian progenitor cells to a downstream lineage of cells. Description of media above may apply to the media (or media components) included in embodiments of the kits. In one embodiment, a kit comprises a first medium. A first medium may be a complete medium, or may be provided as components to be combined by a user. In one embodiment, a kit comprises a first basal medium and a first supplement to be added to the first basal medium. In one embodiment, the first supplement comprises at least one first growth factor. In one embodiment, the at least one first growth factor is a TGF superfamily member. In one embodiment, the at least one first growth factor is an agonist of TGF signaling or BMP signaling. In one embodiment, the at least one first growth factor is TGF-β. In one embodiment, the at least one first growth factor is TGF-β1, TGF-β2, or TGF-β3. In one embodiment, the at least one first growth is GDF-5.

Kits of this disclosure may further comprise a second medium. A second medium may be a complete medium, or may be provided as components to be combined by a user. In one embodiment, a kit comprises a basal medium (whether the same as the first basal medium or different) and a second supplement to be added to the basal medium. In one embodiment, the second supplement comprises at least one second growth factor. In one embodiment, the at least one second growth factor is a TGF superfamily member. In one embodiment, the at least one second growth factor is an agonist of BMP signaling. In one embodiment, the at least one second growth factor is a TGF superfamily member that is different from the at least one first growth factor. In one embodiment, the at least one second growth factor is GDF-5.

In one embodiment, kits comprise a basal medium, a first supplement comprising an agonist of TGF signaling and/or an agonist of BMP signaling, and optionally a second supplement comprising an agonist of BMP signaling.

Supplements included in kits of this disclosure, may be used in different ways depending on the type of cells to be derived from the mammalian progenitor cells. In one embodiment, a first supplement and a second supplement (or a single supplement comprising the instructive growth factors) may be combined in a medium to yield chondrocytes or chondrocyte-like cells. In one embodiment, a first supplement or first medium (as described herein) may be used to culture (and fate) mammalian progenitor cells, followed by a second supplement or second medium (as described herein) to culture (or bias) the arising cells to yield tenocytes or tenocyte-like cells. In one embodiment, tenocytes or tenocyte-like cells are differentiated in only a first medium.

In one embodiment, kits further comprise other growth factors (in the first, second, or additional supplements) that support tenogenic or chondrogenic induction and/or differentiation.

Kits of this disclosure may support serum-free and/or ACF workflows.

In one embodiment, kits further comprise instructions for differentiating mammalian progenitor cells to downstream lineages of cells, as described above.

### Methods

In one aspect of this disclosure are provided methods for differentiating mammalian progenitor cells to downstream lineages, such as the tenogenic lineage or the chondrogenic lineage. In one embodiment, methods of this disclosure are in vitro methods. In one embodiment, methods of this disclosure are performed under serum-free conditions. In one embodiment, methods of this disclosure are performed under ACF conditions.

Mammalian progenitor cells to be contacted by or cultured in media of this disclosure are not particularly limited, and may correspond to any cell type that may be differentiated to a lineage of interest, such as the tenogenic or chondrogenic lineage. Differentiation efficiencies may be enhanced if the mammalian progenitor cells and the target cell share a common germ layer origin. Description of mammalian progenitor cells herein are incorporated into this ‘Methods’ subsection.

Regardless of the source of mammalian progenitor cells (e.g. hBM-MSCs, hADSCs, hTDCs, PSC-derived, such as MPCs) they may be seeded as single cells, small clumps of cells, or at a clonal density. In one embodiment, mammalian progenitor cells are seeded as single cells. In one embodiment, mammalian progenitor cells are seeded at a range of about 3,000-35,000 cells/cm², about 5,000-30,000 cells/cm², about 10,000-25,000 cells/cm², or about 15,000-20,000 cells/cm². The skilled person will know that the seeding density may be adjusted depending on whether clonality is desired (e.g. if the cells have been gene/genome edited beforehand) or on the speed at which the seeded cells are ready to be exposed to differentiation conditions. In one embodiment, mammalian progenitor cells are initially cultured to confluence or subconfluence in expansion conditions, such as in an expansion medium of this disclosure (e.g. MesenCult™-or StemDiff™-branded media). In one embodiment, an expansion medium is a basal medium, such as MesenCult™ ACF Plus, that does not comprise the at least one first and/or the at least one second growth factor(s). Basal media may nevertheless be supplemented with albumin(s), such as recombinant human albumin(s), and other components as described above. In one embodiment, expansion media may comprise one or more growth factors (e.g. at least one FGF).

Methods of this disclosure may comprise culturing mammalian progenitor cells in a first medium, such as after having been seeded and expanded. Description of media hereinabove, including of first media and of second media, are incorporated into this ‘Methods’ subsection.

In one embodiment, first media may comprise a basal medium and at least one first growth factor. As described above, an at least one first growth factor is not particularly limited, as long as it facilitates the differentiation/fating of mammalian progenitor cells, whether to cells of interest or to a cell type intermediate the mammalian progenitor cell and the cells of interest.

In one embodiment, an at least one first growth factor is a TGF superfamily agonist, such as an agonist of TGF signaling and/or an agonist of BMP signaling. In one embodiment, an agonist of TGF signaling and/or an agonist of BMP signaling is a protein or a small molecule.

In one embodiment, an agonist of TGF signaling is an agonist of TGF-β signaling. In one embodiment, an agonist of TGF-β signaling is one of or one or more of TGF-β1, TGF-β2, or TGF-β3. In one embodiment, an agonist of TGF-β signaling is TGF-β3.

In some embodiments, a concentration of a TGF superfamily agonist(s) (e.g. TGF signaling agonist) is between about 0.1 ng/ml and 100 μg/ml, between about 0.5 ng/ml and 10 μg/ml, between about 1 ng/ml and 1 μg/ml, between about 2 ng/ml and 500 ng/ml, between about 3 ng/ml and 100 ng/ml, or between about 5 ng/ml and 10 ng/ml.

In some embodiments, a concentration of TGF-β3 in a first medium (or supplement) is between about 0.1 ng/ml and 100 μg/ml, between about 0.5 ng/ml and 10 μg/ml, between about 1 ng/ml and 1 μg/ml, between about 2 ng/ml and 500 ng/ml, between about 3 ng/ml and 100 ng/ml, or between about 5 ng/ml and 10 ng/ml.

In some embodiments, a concentration of an agonist of BMP signaling (e.g GDF-5) is between about 0.1 ng/ml and 100 μg/ml, between about 0.5 ng/ml and 10 μg/ml, between about 1 ng/ml and 1 μg/ml, between about 2 ng/ml and 500 ng/ml, between about 3 ng/ml and 100 ng/ml, or between about 5 ng/ml and 20 ng/ml.

Methods of this disclosure may also comprise culturing in a second medium those cells arising after culturing mammalian progenitor cells in a first medium. In the alternative, methods of this disclosure may comprise culturing mammalian progenitor cells directly in a second medium to differentiate tenocytes or tenocyte-like cells.

In one embodiment, methods of this disclosure comprise culturing cells or arising cells in a second medium comprising a basal medium and at least one second growth factor. Also as described above, an at least one second growth factor is not particularly limited, as long as it facilitates the differentiation/biasing of an intermediate cell population (e.g. a cell-type downstream of a mammalian progenitor cell) to a cell of interest.

In one embodiment, an at least one first growth factor and an at least one second growth factor are TGF superfamily members. In one embodiment, an at least one first growth factor and an at least one second growth factor are the same TGF superfamily members. In one embodiment, an at least one first growth factor and an at least one second growth factor are different TGF superfamily members. In one embodiment, an at least one second growth factor (e.g. agonist of BMP signaling) is not comprised in a first medium. In the same or different embodiment as the foregoing, an at least one first growth factor (e.g. agonist of TGF signaling) is not comprised in a second (or any) medium.

In one embodiment, an at least one second TGF superfamily member is an agonist of BMP signaling. In one embodiment, an agonist of BMP signaling is a protein or a small molecule. In one embodiment, an agonist of BMP signaling is a growth differentiation factor (GDF) family member. In one embodiment, the GDF family member is GDF-5, GDF-6, GDF-7, or GDF-8. In one embodiment, the GDF family member is GDF-5.

In some embodiments, a concentration of an agonist of BMP signaling (e.g GDF-5) is between about 0.1 ng/ml and 100 μg/ml, between about 0.5 ng/ml and 10 μg/ml, between about 1 ng/ml and 1 μg/ml, between about 2 ng/ml and 500 ng/ml, between about 3 ng/ml and 100 ng/ml, or between about 5 ng/ml and 20 ng/ml.

In one embodiment, a basal medium of a first medium is formulated the same as a basal medium of the second medium. In one embodiment, a basal medium of the first medium is formulated differently from a basal medium of a second medium.

In one embodiment, an at least one first growth factor and an at least one second growth factor may be supplemented sequentially. In one embodiment, an at least one first growth factor and an at least one second growth factor may be supplemented concomitantly.

In one embodiment, culturing in a first medium or in the presence of at least one first growth factor (e.g. agonist of TGF-β or BMP signaling) induces SCX expression, thereby generating cells fated for the tenogenic lineage (which may mature into tenocytes or tenocyte-like cells following continued culture in such conditions). In one embodiment, an at least one second growth factor (e.g. agonist of BMP signaling) induces TNMD expression, thereby generating cells biased for the tenogenic lineage. In one embodiment, culturing in a second medium or in the presence of at least one second growth factor induces SCX and TNMD expression.

Following initial contact with a medium of this disclosure, the culture of cells may or may not be passaged during the differentiation process. In one embodiment, the culture of cells are not passaged after initial contact with a medium of this disclosure (whether a first medium or directly into a second medium). In one embodiment, the culture of cells are not passaged after initial contact with a medium of this disclosure (whether a first medium or directly into a second medium), and also not prior to or after any transition to a different medium of this disclosure (e.g. a second medium). In one embodiment, the culture of cells are passaged either while or after culturing in a first medium, or prior to or following transition of the culture into a second medium.

In one aspect of this disclosure are provided methods of differentiating mammalian progenitor cells to the tenogenic lineage. As described above, such methods may comprise culturing the mammalian progenitor cells in a first medium comprising a basal medium and at least one first growth factor (e.g. agonist of TGF-β signaling) to generate an arising population of cells. In one embodiment, the methods may further comprise culturing the arising population in a second medium comprising a basal medium and at least one second growth factor (e.g. agonist of GDF-5 signaling) to generate tenocytes or tenocyte-like cells. In one embodiment, tenocytes or tenocyte-like cells are differentiated in a single medium formulation comprising a basal medium and at least one growth factor (e.g. agonist of GDF-5 signaling).

The foregoing methods may elapse any time that is sufficient to generate the target cell type (e.g. tenocytes or tenocyte-like cells). In one embodiment, culturing in a first medium may be for between about 1 and 14 days, between about 1 and 7 days, or about 3 days. In embodiments, as described above, where tenocytes are differentiated from mammalian progenitor cells in a single medium, culturing in such medium may be for between about 14 and 28 days, or about 21 days. In embodiments where an arising culture is transitioned to a second culture environment/medium, culturing in the second medium may be for between about 1 and 21 days, between about 7 and 21 days, or between about 14 and 21 days. In one embodiment, culturing in a second medium may be for at least 21 days, such as between about 21 and 42 days, between about 28 and 42 days, or between about 35 and 42 days.

In one aspect of this disclosure are provided methods for differentiating mammalian progenitor cells to the chondrogenic lineage. As described above, such methods may comprise culturing mammalian progenitor cells in a medium comprising a basal medium, at least one first growth factor, and at least one second growth factor to generate chondrocytes or chondrocyte-like cells.

The foregoing methods may elapse any time that is sufficient to generate the target cell type (e.g. chondrocytes or chondrocyte-like cells). In one embodiment, culturing in a medium may be for between about 7 and 42 days, between about 14 and 42 days, between about 21 and 42 days, between about 28 and 42 days, or between about 35 and 42 days. In one embodiment, culturing in a medium may be for between 1 and 42 days, between about 3 and 35 days, between about 7 and 28 days, or about 21 days.

In another aspect of this disclosure are provided methods of differentiating mammalian progenitor cells to a downstream lineage of cells. Such methods may comprise generating a fated cell population by contacting the mammalian progenitor cells with a first medium. In one embodiment, the first medium comprises a basal medium and at least one first growth factor (as described above, such as a TGF superfamily agonist).

The foregoing methods may further comprise biasing the fated cell population to the downstream lineage of cells. Such methods may comprise biasing the fated cell population to the downstream lineage of cells by contacting the fated cell population with a second medium and at least one second growth factor (as described above, such as a TGF superfamily agonist).

In one embodiment, a first medium and a second medium comprise the same TGF superfamily agonist. In one embodiment, a first medium and a second medium comprise different TGF superfamily agonists.

In embodiments where the TGF superfamily agonists are different in each medium, the nature of such agonists may be as described above (e.g. TGF-β signaling agonist and BMP signaling agonist; TM-1>TM-2 as otherwise denoted herein). In embodiments where the TGF superfamily agonists are the same in each medium, the nature of such agonists may be as described above (e.g. BMP signaling agonist). In one embodiment, a first medium does not comprise an at least one second growth factor (e.g. agonist of BMP signaling) and a second medium does not comprise an at least one first growth factor (e.g. agonist of TGF-β signaling).

In one embodiment, the methods may further comprise withdrawing the fated population of cells from contact with a first medium prior to the biasing step.

In one embodiment, methods of this disclosure may further comprise supplementing a first medium comprising a basal medium and at least one first growth factor (and/or a second medium comprising a basal medium and at least one second growth factor) with additional growth factors that support tenogenic or chondrogenic induction/differentiation.

In the foregoing methods the seeding conditions, the passaging conditions (if any), and the duration of the methods may be as described hereinabove.

In one embodiment, the downstream lineage of cells are tenocytes or tenocyte-like cells (TLCs). In one embodiment, the TLC cells express tenogenic markers, such as one or more of SCX, MKX, TNMD, COL1A1, and COL3A1. In one embodiment, the downstream lineage of cells are chondrocytes or chondrocyte-like cells. In one embodiment, the downstream lineage of cells are osteocytes or osteocyte-like cells. In one embodiment, the downstream lineage of cells are adipocytes or adipocyte-like cells.

In any of the methods disclosed herein, including wherein the mammalian progenitor cells are PSC-derived, the mammalian progenitor cells may first be established (e.g. derived and/or expanded) in appropriate conditions before being contacted with/cultured in media of this disclosure (to generate the target downstream lineage of cells). In one embodiment, PSC may be expanded/maintained prior to exposing them to conditions for differentiation to the mammalian progenitor cells. In such an embodiment, the PSCs may be differentiated into MSC-like cells (e.g. mesenchymal progenitor cells or mesodermal precursor cells) using commercially available products/protocols, such as those available under the STEMdiff™ brand. In one embodiment, MSCs, ADSCs, TDCs, or the like from a source tissue may be seeded and expanded in appropriate conditions to expand the culture of cells to confluence or subconfluence (prior to differentiation). In such an embodiment, the cells may be expanded using commercially available products/protocols, such as those available under the MesenCult™ brand.

Methods of this disclosure may be performed under serum-free and/or ACF conditions. In one embodiment, methods of this disclosure are under feeder-free conditions.

Tenocytes or chondrocytes of this disclosure, whether induced/differentiated using media or methods of this disclosure from MSCs, ADSCs, PSCs, or PSC-derived MSCs, may be used to treat various ailments, conditions, disorders, diseases, or injuries, whether cosmetic, medical in nature (e.g. non-surgical), or non-medical nature. For example, the ailments, conditions, disorders, diseases, or injuries may relate to i) musculoskeletal tissue including but not limited to bone, ligaments, tendon, cartilage and the discs of the spine, ii) tendinopathy, tendinosis, tendinitis, tenosynovitis, Achilles tendinitis, patellar tendinitis or lateral epicondylitis, or iii) veterinary applications in horses, cows, sheep, goats, pigs, mice, rats, rabbits, cats or dogs.

Tenocytes or chondrocytes of this disclosure, whether induced/differentiated using media or methods of this disclosure from MSCs, ADSCs, PSCs, or PSC-derived MSCs, may be used to repair or replace biological tissues that have been damaged or rendered dysfunctional due to injury, repetitive use, aging, or otherwise, such as in cosmetic or non-surgical applications.

Accordingly, in the foregoing aspects the tenocytes or chondrocytes, whether induced/differentiated using media or methods of this disclosure from MSCs, ADSCs, PSCs, or PSC-derived MSCs, may be used as a cell transplantation source, such as in autologous or allogeneic procedures.

Where used to treat ailments, conditions, disorders, diseases, or injuries, such as as a cell transplantation source, or to repair or replace biological tissues that have been damaged or rendered dysfunctional due to injury, repetitive use, aging, or otherwise, tenocytes or chondrocytes of this disclosure may be comprised in a composition. In one embodiment, such a composition may be a cell pellet or (micro) tissue. In one embodiment, such a composition may be comprised in a medium or combined with an excipient or scaffold/substrate. Examples of scaffolds/substrates include but are not limited to a matrix, a membrane, a microbead, a fleece, a thread, a gel, or mixtures thereof. In some embodiments, the scaffold may include collagen (Type I, II and/or III) or proteins or polypeptides such as hyaluronic acid, polylactic acids, or polymers such as elastin, fibrin, laminin or fibronectin. In some embodiments, scaffolds/substrates are biological scaffolds derived from mammalian tissues and may be selected from Arthroflex®, Dermaspan™, BioArthro™, TissueMend® or Restore™. In some embodiments, scaffolds/substrates are synthetic polymers and may be selected from polystyrene, poly-I-lactic acid (PLLA), polyglycolic acid (PGA) and poly-dl-lactic-co-glycolic acid (PLGA).

In another aspect of this disclosure are provided media and methods for in-vitro expansion of primary mammalian (e.g. human) tenocytes. In one embodiment, media and methods of this aspect comprise culturing primary mammalian tenocytes in a serum-free and/or ACF workflow/medium. In one embodiment, the medium is a MesenCult™-branded medium (e.g. MACF+, STEMCELL Technologies). In one embodiment, an expansion medium is a basal medium, such as MesenCult™ ACF Plus, not comprising an at least one first and/or at least one second growth factor(s). Basal media, as described herein, may be supplemented with (recombinant) albumin(s), as well as other components, also as described above. In one embodiment, expansion media may comprise one or more growth factors (e.g. at least one FGF).

In one embodiment, media and methods of this aspect support time in (length of) culture and/or population doublings of primary human tenocytes under serum-free and/or ACF conditions that are improved/increased over or consistent with (e.g. ±15% of, ±10% of, or ±5% of) conventional, undefined (bovine) serum-containing conditions. In one embodiment, media and methods of this aspect support population doublings of primary mammalian tenocytes for at least 10 passages, at least 9 passages, at least 8 passages, at least 7 passages, at least 6 passages, at least 5 passages, at least 4 passages, or at least 3 passages, or less. In one embodiment, media and methods of this aspect support population doublings for over 50 days in culture, or about 40 days, about 30 days, or about 20 days.

The following non-limiting examples are illustrative of the present disclosure.

### EXAMPLES

Example 1

### Preparation of Human MSCs, PSCs and Primary TDCs

Human MSCs were isolated and expanded for as many passages as required, but typically were taken for downstream applications after expanding for 3 passages in MesenCult™-ACF Plus Medium Kit (MACF+, STEMCELL Technologies).

Human PSCs were expanded for at least 5 passages in TeSR™-E8™ (STEMCELL Technologies) in a feeder-free and serum-free environment. Human PSC lines were tested for karyotypic abnormalities using the hPSC Genetic Analysis Kit (STEMCELL Technologies), and karyotypes were confirmed as normal before differentiation experiments.

Except for certain control conditions, all MSCs and PSCs used in the below examples were cultured and processed in a completely ACF environment. However, given the lack of an ACF medium for in vitro expansion of primary hTDCs, such cells were isolated and expanded in standard serum-containing culture conditions, consisting of DMEM-F12 (STEMCELL Technologies) supplemented with 10% FBS (Fisher Scientific).

Human tendons were obtained from patients without tendinopathy but were undergoing tendon surgery in accordance with appropriate licenses, ethical approvals, and informed consent in place. The tissues were cleaned aseptically and cut into small pieces and placed into 6-well plates. Tendon segments were supplemented with 5 ml of culture media containing Dulbecco's modified Eagle medium (DMEM), 10% FBS, and 1% penicillin-streptomycin and placed at 37° C. in a humidified atmosphere of 5% CO₂. Culture medium was changed every 3 days. Once migrated tenocytes reached 80-90% confluency, they were treated with EDTA and sub-cultured in T-175 tissue culture flasks.

### Example 2

In vitro Tenogenic Differentiation

Human MSCs derived from bone marrow were plated on cell-culture treated 24-well plates (Corning) at a cell density of 15,000-20,000 cells/cm² in MACF+. Unless stated otherwise, once culture wells reached ˜90% or higher confluence they were washed with PBS and transitioned to tenogenic differentiation conditions: in TGF-β3 for 3 days (stage 1 medium, or TM-1); and from day 3 onward in GDF-5 (but not TGF-β3) (stage 2 medium, or TM-2). The sequential protocol may be referred to as TM-1>TM-2. Medium changes were performed every other day until day 21 when the cells were characterized.

In order to generate cells with tenocyte-like properties from human iPSCs or ESCs in an ACF environment a stepwise differentiation method was adopted, wherein the PSCs were initially differentiated to MPCs in STEMdiff™ Mesenchymal Progenitor kit (STEMCELL Technologies) for 21 days, as recommended by the manufacturer, then the MPC cultures were transitioned into a tenogenic differentiation protocol as described above.

### Example 3

Evaluation of Tenogenic Differentiation by qPCR, ICC and Flow Cytometry

Tenocyte-like cells derived in accordance with Example 2 were evaluated by quantitative PCR (qPCR), immunocytochemistry (ICC), and flow cytometry.

For qPCR analysis, total RNA was extracted with RNeasy Mini Kit (QIAGEN) and quantified using Nanodrop 2000 (Thermo Fisher Scientific). Reverse transcription was carried out with High Capacity cDNA Reverse Transcription Kit (Applied Biosystems). Quantitative real-time PCR was performed using the StepOne Plus Real-Time PCR system (Life Technologies). Standard curves were generated for each target. Gene expression levels are normalized to housekeeping genes (TBP or 18S) and plotted as log2 fold change values, obtained by calculating the logarithm to the base 2 of the difference between the average of the Ct values for the housekeeping gene and the average of the Ct values for the gene of interest.

On the day of ICC, cells were: washed 2× with PBS; fixed in 4% PFA diluted in PBS; permeabilized with Triton-X 100 0.2% in PBS for 10 minutes at room temperature; exposed to 3% BSA in PBS blocking solution for 60 minutes; incubated with primary antibodies at specific dilutions for each target (ranging from 1:50 to 1:400) for 60 minutes at room temperature; incubated with a secondary antibody (dilution 1:400) in the dark for 60 minutes; and counterstained for 1 minute with DAPI 3 μM (STEMCELL Technologies). Cultures were observed with a Leica DMi8 inverted fluorescence microscope.

Surface marker expression was assessed by flow cytometry (CytoFlex, Beckman Coulter; CytExpert Software, Beckman Coulter) using conjugated antibodies as follows: PE anti-human CD45 (cat. no. 304007); BV421 anti-human CD73 (cat. no. 344007); FITC anti-human CD90 cat. no. 328107); APC anti-human CD105 (cat. no. 800507); and Zombie NearIR fixable viability stain (cat. no. 423105). Briefly, cells were detached using TrypLE™ Express Enzyme (1×) (Thermo Fisher Scientific, cat. no. 12605010) and resuspended in DMEM +2% FBS. The cell suspension was centrifuged at 1500 rpm for 5 minutes and washed three times with PBS +0.2% BSA before incubating with antibody solution for 45 minutes in the dark at room temperature in PBS+0.2% BSA. For all tubes, at least 50,000 cells were analysed.

### Example 4

Supplementation of Media with Tendon-Inducing Factors

MSCs were obtained as described in Example 1, plated in cell-culture treated 24-well plates as described in Example 2, and exposed to the following culture conditions: i) TGF-β3 alone (e.g. 10 ng/ml), ii) GDF-5 alone (e.g. 20 ng/ml), iii) TGF-β3 (e.g. 10 ng/ml) and GDF-5 (e.g. 20 ng/ml), iv) TGF-β3 (e.g. 10 ng/mL) followed by GDF-5 (e.g. 20 ng/ml), and v) control condition (cells in MACF+). After 21 days in culture at full confluence, the cells were analyzed as described in Example 3 by qPCR for markers of early tenocytes (SCX and MKX) and mature tenocytes (TNMD and COL1A1), and also by ICC.

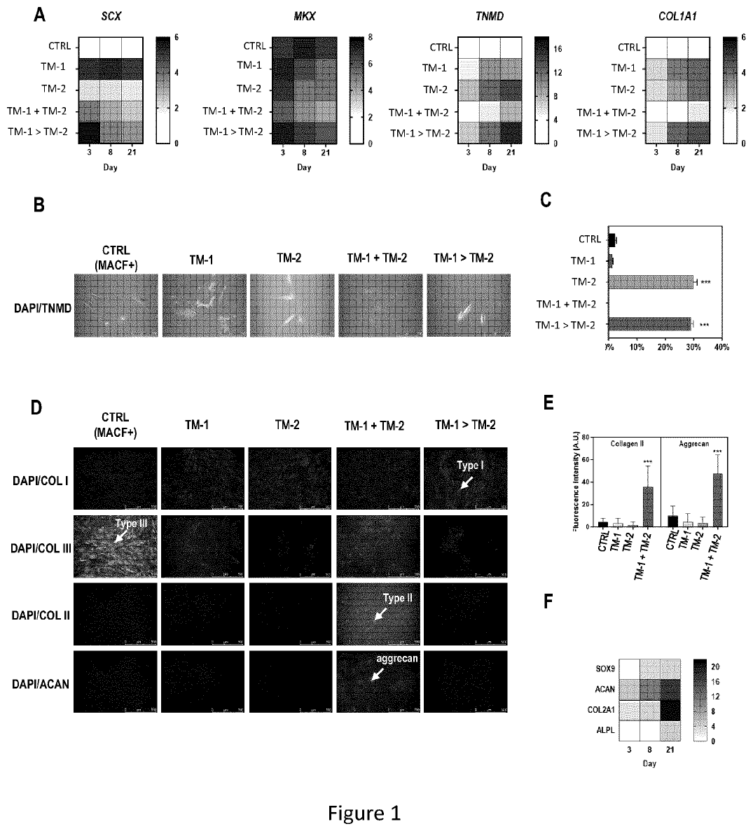

Time-course qPCR showed distinct trends in tenogenic marker expression among the tested culture conditions (FIG. 1A).

SCX is a transcription factor required for tenogenic lineage commitment of mesenchymal progenitors/stem cells. Exposure to TGF-β3 alone consistently increased SCX mRNA levels at an early time point (day 3), and SCX mRNA levels remained elevated for the duration of the experiment (until day 21). In contrast, expression of SCX remained low when cells were exposed to GDF-5 alone. Where both TGF-β3 and GDF-5 were combined for the duration of the experiment, limited induction of SCX mRNA occurred by day 3 which steadily decreased at day 8 and day 21. However, when TGF-β3 and GDF-5 were added sequentially, SCX mRNA levels at day 3 were comparable to the TGF-β3 alone condition, but gradually became reduced over the course of tenocyte maturation. MKX, another essential regulator of tenogenic differentiation and mechanosensing, was most highly expressed at day 3 and slightly decreased at subsequent time points in all conditions. In contrast, MKX expression levels were consistently high in the control condition.

TNMD, a marker of mature differentiated tenocytes, showed significant upregulation in the GDF-5 alone and in the sequential supplementation conditions. In contrast, the control condition resulted in little to no expression of TNMD mRNA, while the TGF-β3 alone and the combined TGF-β3+GDF-5 condition exhibited only modest expression of TNMD mRNA. Collagen type I α1 (COL1A1), another marker of mature tenocytes, became upregulated throughout tenogenic differentiation in all formulations, except where TGF-β3 and GDF-5 were supplemented simultaneously (FIG. 1A).

Phenotypes of the cells arising out of the tested cultured conditions was assessed by ICC. MSCs differentiated in either GDF alone or in GDF-5 after an initial TGF-33 exposure exhibited notable TNMD protein expression throughout the cell body and an absence of contractile stress fibers (FIG. 1B), in comparison to the other tested conditions. Moreover, around 30% of cells cultured in these conditions adopted an elongated and spindle-shaped morphology characteristic of mature tenocytes (FIG. 1C). In contrast, MSCs differentiated in TGF-β3 alone or in TGF-β3 and GDF-5 combined resembled myofibroblasts having a flattened and stretched morphology, appearance of actin stress fibers, and low TNMD expression (FIG. 1B and 1C). In fact, the cell monolayer in the TGF-β3 alone condition exhibited ECM contraction as early as day 7, which became more pronounced by day 21. In contrast, hMSCs maintained at high density in ACF tenogenic medium persisted as a well-attached uniform cell monolayer sheet on 24-well plate and 6-well plate culture wells (not shown).

In addition to the induction of pro-tenogenic genes and the acquisition of typical features of mature tenocytes, such as expression of TNMD and the acquisition of an elongated spindle-shaped morphology, an important characteristic of differentiated tenocytes is the ability to synthesize extensive amounts of collagen type I in the extracellular environment. Thus, collagen type I and type III deposition during the differentiation of hMSCs in the culture conditions of this Example 4 was examined by ICC. Proliferating and undifferentiated hMSCs at day 0 did not exhibit apparent deposition of collagen type I or type III (data not shown) consistent with the low mRNA expression shown in FIG. 1A. As shown in FIG. 1D, MSCs in the control condition deposited an ECM matrix predominantly composed of collagen type III, and trace amounts of collagen type I. During tenogenic differentiation in cultures supplemented with TGF-β3 alone, GDF-5 alone, and with GDF-5 after an initial exposure to TGF-β3, the ECM was predominantly composed of collagen type I. Collagen type III protein became barely detectable at day 21 in the sequential condition, comparable to the TGF-β3 alone or the GDF-5 alone culture conditions. On the contrary, the combined supplementation of TGF-β3+GDF-5 induced the deposition of a matrix mainly composed of collagen type II and aggrecan (FIG. 1D), as confirmed by densitometry (FIG. 1E). Additional qPCR analysis on chondrogenic markers (SOX9, ACAN, COL2A1, ALPL) revealed that the combined supplementation of TGF-β3+GDF-5 appeared to induce chondrogenic differentiation of MSCs (FIG. 1F).

Therefore, among the tested tenogenic formulations, the sequential exposure to TGF-β3 followed by GDF-5 desirably induces the timely expression of early (SCX, MKX) and late (TNMD) tenogenic markers, culminating with the generation of elongated spindle-shaped TNMD+ cells producing extensive amounts of collagen type I in only three weeks.

### Example 5

Differentiating hBM-MSCs and hTDCs Towards Tenogenic Lineage

The differentiation potential to the tenogenic lineage of various starting cell types was next explored. MSCs and TDCs were obtained as described in Example 1. Each cell type was cultured in the TM-1->TM-2 condition, essentially as described in Example 2. As in Example 4, features of the arising cells were evaluated by qPCR, histology and ICC.

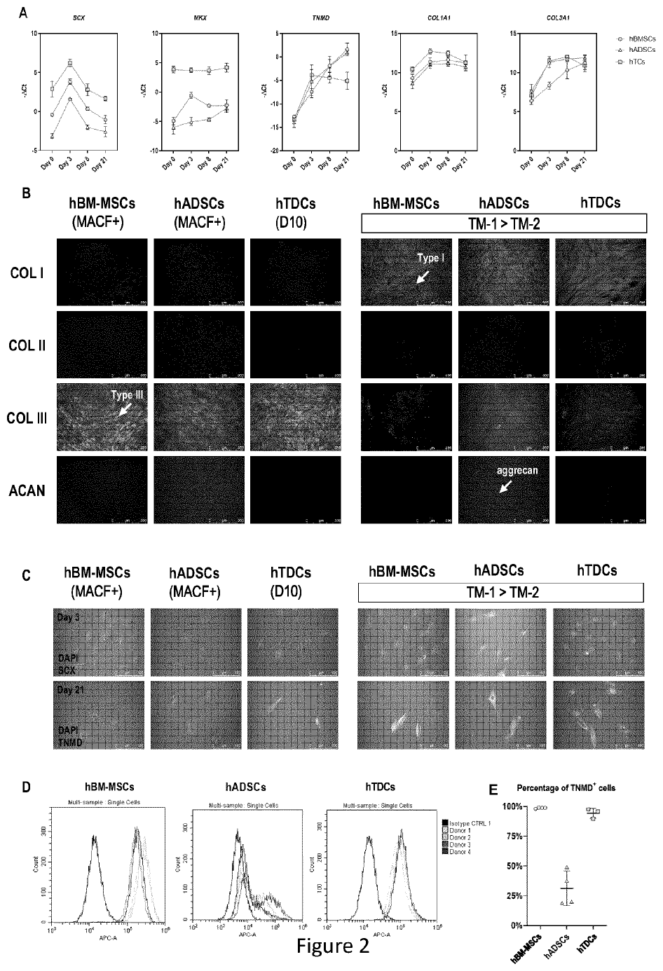

Time-course qRT-PCR on day 0, 3, 8 and 21 showed that tenogenic lineage markers were upregulated in hBM-MSCs and hTDCs cultured in TM-1>TM-2 (FIG. 2A).

Representative immunofluorescence (10× magnification) images of tenogenic cultures stained for collagen type I, type II, type III, and aggrecan demonstrated that the hBM-MSCs and hTDCs cells deposited an ECM rich in collagen type I when exposed to TM-1>TM-2, and produced less collagen type III compared to the control condition (FIG. 2B).

ICC analysis of several tenogenic (SCX, TNMD, COL1, COL3), chondrogenic (COL2, Aggrecan) and myofibroblast (αSMA) markers was performed. Representative immunofluorescence images at 40× magnification of tenogenic cultures stained for SCX and TNMD at day 3 and day 21 respectively showed marked differentiation of hBM-MSCs and hTDCs cells toward the tenogenic lineage in the TM-1>TM-2 condition, as compared to limited or no SCX and TNMD expression in the control conditions (hBM-MSCs and HADSCs in MACF+; and hTDCs in DMEM+10% FBS (D10)) (FIG. 2C).

Next, the cells were characterized by flow cytometry for the expression of relevant surface markers. The flow cytometry histograms showed a significant increase of TNMD+ cells among hBM-MSCs and hTDCs differentiated in TM-1>TM-2 (FIG. 2D and 2E). These results indicate that the methods and media of this disclosure efficiently differentiate hBM-MSCs, and hTDCs towards the tenogenic lineage.

### Example 6

### Extended Passage Culture of Primary Human Tenocytes

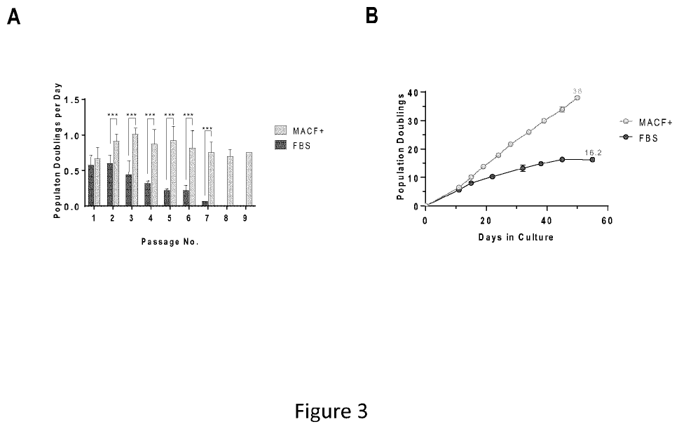

Primary human tenocytes are routinely cultured in FBS-containing media, which results in rapid phenotype loss, downregulation of tenogenic markers, loss of spindle-shaped morphology, and limited expandability until P3-P5. Primary human tenocytes were obtained as described in Example 1 and cultured in either MACF+ or FBS-based culture conditions. Proliferation of cells in either medium formulation was assessed, and population doublings steadily decreased in the FBS-containing formulation (FIG. 3A and 3B). In contrast, the number of population doublings was steady for at least 9 passages in MACF+ (FIG. 3A), and the number of population doublings increased steadily over more than 50 days in culture (FIG. 3B). In addition, qPCR analysis performed during the first 5 passages on cultures in both formulations revealed that expression of tenogenic markers among hTDCs expanded in MACF+ did not decrease over time in culture (data not shown).

Collectively these data suggest that primary human tenocytes can be efficiently expanded in MACF+ medium, while overcoming the numerous problems associated with their culture in FBS-containing media.

### Example 7

hiPSCs and hESCs are Differentiated into Cells with Tenocyte-Like Properties

It is unknown whether hPSC-derived MSCs (mesenchymal progenitor cell, “MPC”) have the capacity to differentiate to tenocyte-like cells (“TLC”s). Thus, stepwise differentiation as described in Example 2 was conducted on PSCs maintained as described in Example 1.

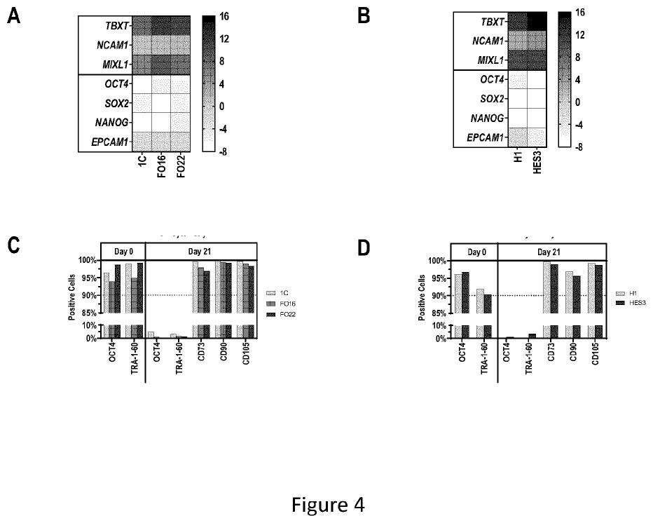

First, MPCs were generated from 3 different iPSC lines (WLS-1C, F016, and F022) and 2 different ESC lines (H1 and HES3) in accordance with Example 2, and the MPCs output after the 21-day differentiation were analyzed by quantitative PCR for expression of mesoderm markers (TBXT, NCAM1, and MIXL1) and pluripotency markers (OCT4, SOX2, NANOG, and EPCAM1). MPCs derived from hiPSCs (FIG. 4A) and hESCs (FIG. 4B) showed increased expression of mesoderm markers and reduced expression of pluripotency markers. Flow cytometry analysis revealed that fewer than 10% of day-21 MPCs differentiated from the iPSC and ESC lines expressed pluripotency markers OCT4 and TRA-1-60 (FIGS. 4C and 4D, respectively). Further, >90% of MPCs differentiated from iPSC and ESC lines express typical hMSC markers, such as CD73, CD90, and CD105 (FIGS. 4C and 4D).

In the next stage of differentiation, MPCs were transitioned into the stepwise tenogenic differentiation protocol as described in the Example 2. Similar to earlier examples, tenogenic differentiation of PSC-derived MPCs was evaluated by qPCR, histology and ICC.

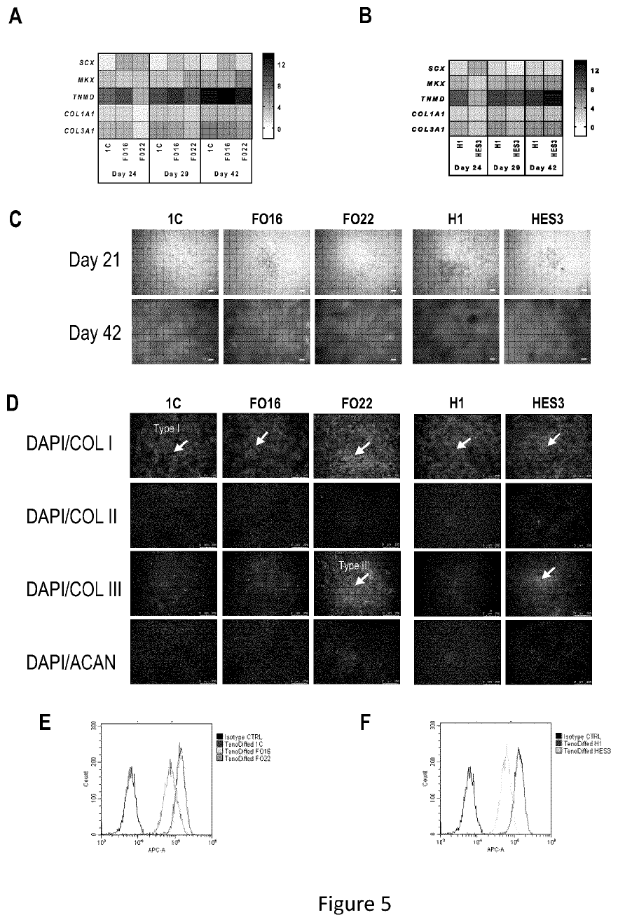

Heat maps of qPCR analysis at day 3 (day 24), day 8 (day 29) and day 21 (day 42) showed upregulation of the main tenogenic markers SCX, MKX, TNMD, COL1A1 and COL3A1 for MPC derived from each hiPSC (FIG. 5A) and hESC (FIG. 5B) line tested.

MPCs cultured in TM-1>TM-2 were stained with Sirius Red at the start (day 21) and the end (day 42) of tenogenic differentiation. Extensive collagen deposition was shown for all PSC-derived tenocyte-like cell cultures (FIG. 5C). To clarify the nature of the collagen deposition, representative immunofluorescence images were taken at 10× magnification of cultures stained for collagen type I, type II, type III, and aggrecan. All PSC-derived tenocyte-like cell cultures consistently deposited a tendon-like ECM predominantly composed of collagen type I with, in some cases, traces of collagen type III (FIG. 5D).

The expression of TNMD at day 21 after MPCs were differentiated in TM-1>TM-2was evaluated by flow cytometry and the histograms in FIGS. 5E and 5F indicate for each starting PSC cell line that more than 95% of differentiated cells expressed TNMD.

Collectively these data indicate that the described tenogenic workflow robustly and efficiently differentiated not only MSCs isolated from different sources, but also performed consistently on MPCs derived from PSCs.

### Example 8

### RNA-seq Reveals the Activation of a Tenogenic Transcriptional Program During Differentiation of MSCs and PSCs

To better characterize and explore the transcriptomes of the TLCs derived from hMSCs and hiPSCs in TM-1>TM-2 (TenoDiffed MSCs and TenoDiffed hiPSCs, respectively), a global RNA-seq analysis was performed. Results were compared to the starting cell type and to hTDCs expanded in vitro for 3 passages in MACF+ medium.

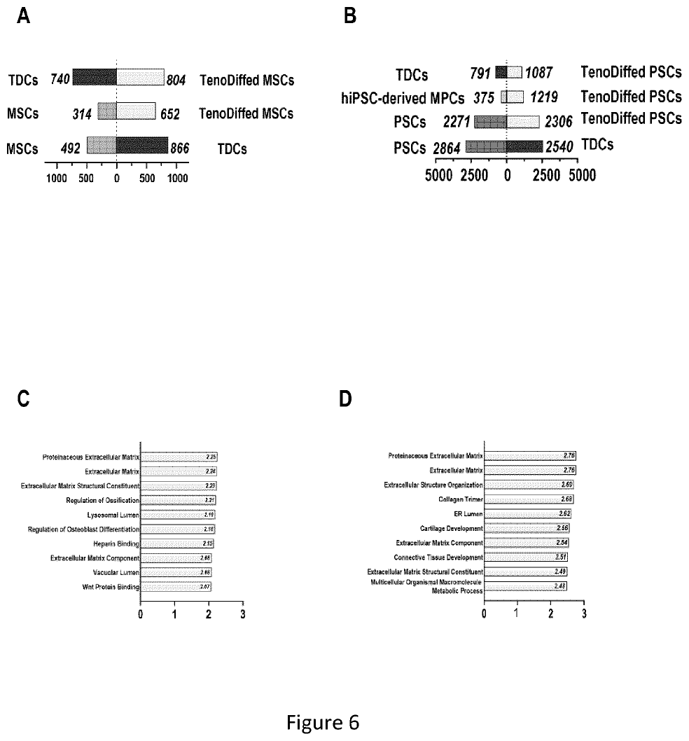

The analysis showed 1,444 differentially expressed genes (“DEGs”, at least 2-fold difference and a p-value less than or equal to 0.01) between TDCs and TenoDiffed MSCs, 966 DEGs between MSCs and TenoDiffed MSCs, and 1,358 DEGs between MSCs and TDCs (FIG. 6A). In order to correlate gene associations with certain biological processes or molecular functions, the DEGs were categorized by Kyoto Encyclopaedia of Genes and Genomes (KEGG) pathway analysis. Comparing MSCs to TenoDiffed MSCs revealed that pathways involved in tenogenic differentiation were upregulated, such as TGF-β and PI3K, along with Rap1 signaling and protein digestion and absorption (data not shown). Next, the molecular and cellular functions of the DEGs between TenoDiffed MSCs and the starting cell population were examined by a gene set enrichment analysis (GSEA) on the Gene Ontology (GO) network. The GSEA confirmed that the most positively enriched gene sets in TenoDiffed MSCs were related to the synthesis of ECM components, skeletal system development, and connective tissue development (FIG. 6C).

The same approach evaluated transcriptomic differences induced during tenogenic differentiation of hPSCs. The analysis revealed that the differences between TDCs and differentiated PSCs (TenoDiffed PSCs) were substantially lower than the differences between either PSCs and TDCs, or PSCs and TenoDiffed PSCs (FIG. 6B). Indeed, only 1,878 DEGs between TDCs and TenoDiffed PSCs were identified, compared to 4,577 DEGs between PSCs and TenoDiffed PSCs, and 5,404 DEGs between PSCs and TDCs (FIG. 6B). A KEGG pathway analysis highlighted the activation of several tenogenic pathways in TenoDiffed PSCs—such as TGF-β, PI3K-AKT, MAPK—together with pathways related to cell adhesion and synthesis of ECM components (data not shown). In contrast, genes upregulated in PSCs clustered in pathways involved in cell cycle progression and neural development, indicating a primitive phenotype (data not shown). GSEA using the C5 MSigDB database confirmed that synthesis of ECM components, skeletal system development, and connective tissue development were the most positively enriched gene sets in TenoDiffed PSCs compared to PSCs (FIG. 6D).

Collectively, these data suggest that the cells differentiated using media and methods of this disclosure (e.g. TM-1>TM-2) exhibit an activation of a tenogenic transcriptional program when starting from either MSC or hPSC.

### Example 9

### Consistent Gene Expression Signatures of Tenogenic Cells and Various Starting Cells

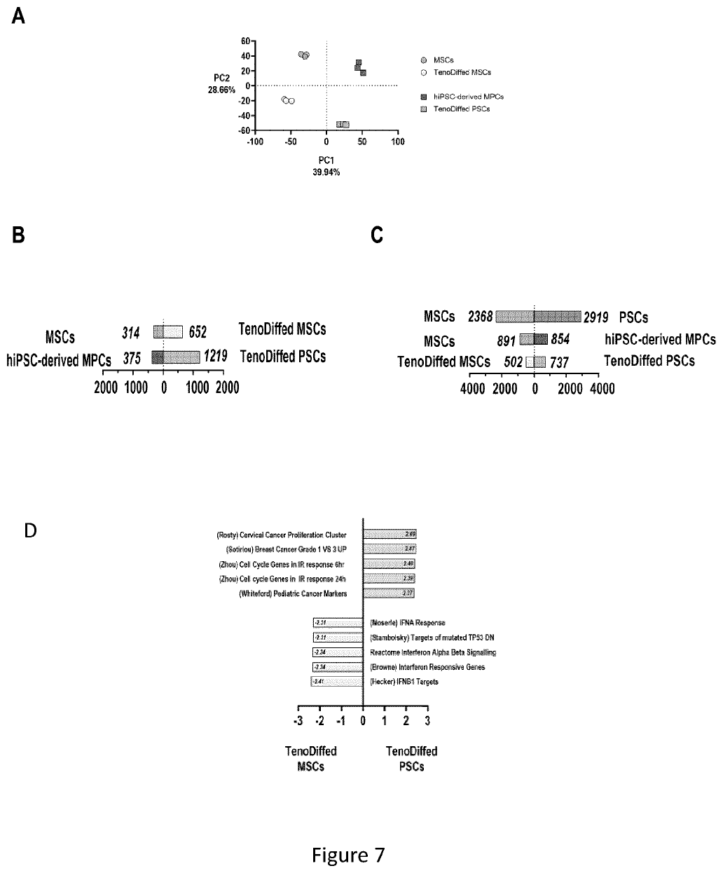

MSCs and PSCs were differentiated as described in Example 2 and their transcriptional signatures were compared to better understand the importance of the stem cell source on tenogenic differentiation (FIG. 7). A principal component analysis indicates i) a separation of MSC and differentiated MSCs, from hPSC-derived cells along PC1 (39.94% of the variance), and ii) undifferentiated cells are separated from differentiated cells along PC2 (28.66% of the variance) (FIG. 7A). These data suggest that (i) the transcriptomic differences seen between the TenoDiffed cells occur at the MSC derivation stage, and (ii) the genes involved in transitioning from MSCs to TenoDiffed MSCs are similar to those transitioning from hiPSC-derived MPCs to TenoDiffed PSCs. Differential gene expression analysis (2 fold difference and a p-value less than or equal to 0.01) was performed to confirm these hypotheses and the data revealed similar transcriptomic differences between the differentiated tenogenic cells and their respective starting cells (FIG. 7B). Strikingly, 49.5% of the DEGs between MSCs and TenoDiffed MSCs (478 out of 966) were also differentially expressed when comparing hiPSC-derived MPCs and TenoDiffed PSCs. A subset of these genes correlated with upregulation of tenogenic pathways, as well as with gene sets associated with ECM synthesis and organization (data not shown). These data reveal that tenogenic differentiation of this disclosure induces similar tenogenic transcriptional programs across different starting stem cell populations.

The transcriptional differences at different stages of differentiation were further assessed. While 5,287 DEGs were identified between the PSC and MSC populations, the number of DEGs decreased to 1,745 between MSCs and MPCs, and to 1,239 between TenoDiffed MSCs and TenoDiffed PSCs (FIG. 7C). Of the differences between TenoDiffed MSCs and TenoDiffed PSCs, the former showed upregulation of multiple interferon gene sets while the latter showed the enrichment of gene sets related to cancer and cell cycle progression (FIG. 7D). Notably, more than half of the DEGs between TenoDiffed MSCs and TenoDiffed PSCs (626 out of 1,239) were also differentially expressed between MSCs and hiPSC-derived MPCs. These pre-existing differences were mainly correlated to binding of calcium ion, lipids and heparin, as well as collagen formation, interferon signaling and arachidonic acid metabolism (data not shown).

These data confirmed that half of the differences seen between TenoDiffed MSCs and TenoDiffed hPSCs are accounted for by the derivation of MPCs, and that the tenocyte differentiation portion of the protocol accounts for comparatively few transcriptomic differences between the starting stem cell populations.

### Example 10

### Similar Marker Expression Among TenoDiffed Cells and Primary Tendon Cells

Transcriptional programs of TenoDiffed cells with those of in vitro-expanded hTDCs and primary Achilles tendon tissue samples were compared. MSC-and PSC-derived tenogenic cells were obtained and cultured as described in Examples 1 and 2, and RNA-seq was performed. Differential gene expression analysis showed that TDCs and TenoDiffed cells shared 1,224 upregulated DEGs and 1,807 downregulated DEGs (data not shown). Upregulated DEGs implicated ontologies related to cell cycle progression and energy metabolism, indicating a quiescent phenotype for the resident cell populations of the tendon tissue. Downregulated DEGs implicated ontologies related to innate and acquired immunity, revealing an active immune response taking place in non-expanded primary cells (data not shown).

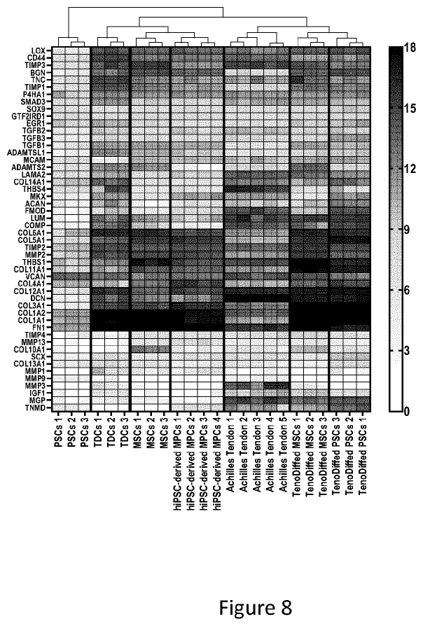

To explore the foregoing transcriptional signatures in more detail, the expression profiles of 50 genes related to tenogenic differentiation, tendon development and tendinopathies were examined across the same samples (FIG. 8). The gene expression profiles of TenoDiffed MSCs and TenoDiffed PSCs showed a uniform upregulation of tenogenic-related genes comparable to non-expanded primary Achilles tendon cells, in particular those genes related to collagen deposition and ECM generation (FIG. 8). Conversely, tenogenic gene expression among in vitro-expanded TDCs were more similar to MSCs and hiPSC-derived MPCs. Finally, PSCs showed very low expression levels of tenogenic markers, and therefore clustered separately from all other cell types in the study. Most genes of the collagen family (namely COL1A1, COL1A2, COL3A1, COL4A1, COL5A1, COL6A1, COL11A1, COL12A1) were highly expressed in all MSC-like and tenocyte-like cells.

### Example 11

### Tenogenic Differentiation of Equine MSCs and SDFTs

Equine MSCs were isolated from bone marrow, plated out into T25 plastic tissue culture flasks with added Dulbecco's Modified Eagles Medium (DMEM) containing 10% fetal calf serum, 100 units/ml penicillin and 0.1 mg/ml streptomycin, 0.11 mg/ml sodium pyruvate and 1% glutamine. After 24-72 h, the unattached cells were washed off with PBS and fresh media added. Adherent cells were cultured to ˜85-90% confluency then passaged into T225 flasks.

Equine SDFTs were isolated from horse forelimbs, digested with collagenase and filtered. Resulting cells were cultured in DMEM supplemented with 10% FBS, with twice weekly medium changes. When culture wells reached >90% confluence, cells were differentiated toward tenogenic lineage as described in Example 2.

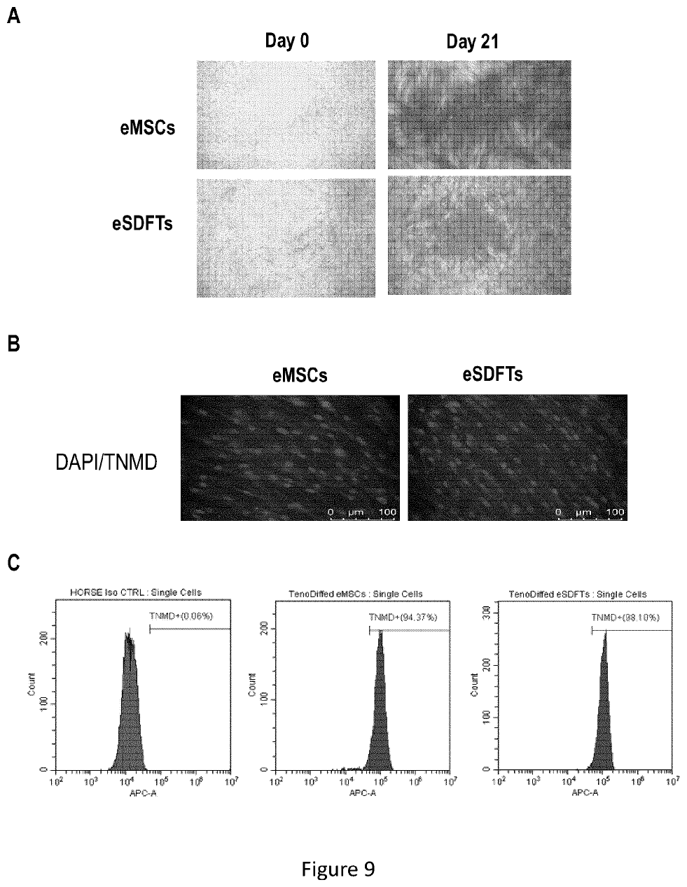

Arising eMSCs and eSDFTs cultures were stained with Sirius Red at day 0 and at the end (day 21) of tenogenic differentiation. Day 21 cells showed extensive deposition of collagen (FIG. 9A). At the protein level, both the eMSCs and eSDFs cells expressed TNMD protein at day 21 as shown by the representative immunofluorescence images (FIG. 9B). Flow cytometry analysis indicated that 94-98% of cells are TNMD+ at the end of differentiation (FIG. 9C).

Collectively, these data show that the disclosed media and methods appear to differentiate equine cells to the tenogenic lineage.

1. A method of differentiating mammalian progenitor cells to a downstream lineage of cells, comprising culturing the mammalian progenitor cells in a basal medium comprising at least one first growth factor and optionally at least one second growth factor,
wherein the downstream lineage of cells are tenocytes or tenocyte-like cells, or the downstream lineage of cells are chondrocytes or chondrocyte-like cells.

2-3. (canceled)

4. The method of claim 1, further comprising
(a) deriving the tenocytes or tenocyte-like cells in the presence of the at least one second growth factor sequentially after exposure to the at least one first growth factor; or
(b) deriving the chondrocytes or chondrocyte-like cells when the basal medium comprises the at least one first growth factor and the at least one second growth factor.

5-7. (canceled)

8. The method of claim 1, wherein the at least one first growth factor and the at least one second growth factor are transforming growth factor (TGF) superfamily members.

9. The method of claim 8, wherein the at least one first growth factor is different from the at least one second growth factor.

10. The method of claim 8, wherein the at least one first growth factor is an agonist of TGF-β signaling.

11. The method of claim 10, wherein the agonist of TGF-β signaling is TGF-β1, TGF-β2, or TGF-β3, preferably TGF-β3.

12. (canceled)

13. The method of claim 8, wherein the at least one second growth factor is an agonist of BMP signaling.

14. The method of claim 13, wherein the agonist of BMP signaling is GDF-5.

15. A method of differentiating mammalian progenitor cells to a downstream lineage of cells, comprising:
a) generating a fated cell population by contacting the mammalian progenitor cells with a first medium comprising a basal medium and a TGF superfamily agonist; and
b) biasing the fated cell population to the downstream lineage of cells by contacting the population with a second medium comprising a basal medium and a TGF superfamily agonist.

16. The method of claim 15, wherein;
(i) the TGF superfamily agonist is the same in steps a) and b); or
(ii) the TGF superfamily agonist is different in steps a) and b).

17. (canceled)

18. The method of claim 16, wherein the TGF superfamily agonist is an agonist of TGF-β signaling.

19. The method of claim 18, wherein the agonist of TGF-β signaling is TGF-β1, TGF-β2, or TGF-β3, preferably TGF-β3.

20. (canceled)

21. The method of claim 167, wherein the TGF superfamily agonist in the first medium is TGF-β1, TGF-β2, or TGF-β3, preferably TGF-β3, and the TGF superfamily agonist in the second medium is an agonist of BMP signaling, wherein the first medium does not contain the agonist of BMP signaling and the second medium does not contain the agonist of TGF signaling.

22. The method of claim 21, wherein the agonist of BMP signaling is GDF-5.

23. The method of claim 21, wherein the downstream lineage of cells are tenocytes or tenocyte-like cells.

24-25. (canceled)

26. The method of claim 1, wherein the mammalian progenitor cells are:
(a) MSCs, PSCs, mesenchymal progenitor cells, ADSC, or tendon-derived cells (TDC); and/or
(b) PSC-derived MSC, PSC-derived MPCs, or PSC-derived mesenchymal progenitor cells; and/or
(c) are of human origin, equine origin, rodent origin, porcine origin, bovine origin or canine origin.

27-32. (canceled)

33. A kit for differentiating mammalian progenitor cells to i) tenocytes or tenocyte-like cells, and/or ii) chondrocytes or chondrocyte-like cells, comprising:
a) a basal medium;
b) a first supplement comprising an agonist of TGF signaling; and
c) a second supplement comprising an agonist of BMP signaling.

34. The kit of claim 33, wherein the agonist of TGF signaling is an agonist of TGF-β signaling, and the agonist of TGF-β signaling is TGF-β1, TGF-β2, or TGF-β3, preferably TGF-β3.

35-36. (canceled)

37. The kit of claims 33, wherein the agonist of BMP signaling is GDF-5.

38. (canceled)
